(19) United States
(12) Patent Application Publication
(10) Pub. No.: US 2017/0347689 A1
(43) Pub. Date: Dec. 7, 2017

(54) A PROBIOTIC-FERMENTED MACA COMPOSITION AND A METHOD FOR PREPARING THE COMPOSITION AND USE OF THE COMPOSITION

(71) Applicants: Bingxin WU, Shandong Province (CN); Xiaolin SUN, Shandong Province (CN); Jichen LU, Shandong Province (CN); Xinhong CHU, Shandong Province (CN); Zongwu ZHEN, Shandong Province (CN)

(72) Inventors: Bingxin WU, Shandong Province (CN); Xiaolin SUN, Shandong Province (CN); Jichen LU, Shandong Province (CN); Xinhong CHU, Shandong Province (CN); Zongwu ZHEN, Shandong Province (CN)

(73) Assignee: Bingxin WU, Shandong (CN)

(21) Appl. No.: 15/538,900

(22) PCT Filed: Sep. 9, 2015
(86) PCT No.: PCT/CN2015/000634

§ 371 Date: Jun. 22, 2017

(30) Foreign Application Priority Data
Jan. 14, 2015 (CN) 201510017222.9

(51) Int. Cl. A23L 2/38; A23L 2/84; A61K 35/747; A61K 35/745; A23L 33/00

(52) U.S. Cl. CPC A23L 2/38; A61K 35/747; A61K 35/745; A23L 2/84; A23L 33/00

(57) ABSTRACT

Provided are a probiotic-fermented maca composition, a preparation method therefor, and use thereof. The composition is prepared from maca, fructus lycii and fructus rubi by milling, water extraction, enzymolysis and fermentation by Bifidobacterium and Lactobacillus, and can be used for preparing foods, healthcare products or beverages for improving fatigue tolerance and sexual function.

### TECHNICAL FIELD

The present invention relates to a maca composition fermented by one or more bowel probiotics and a method for preparing the composition, and belongs to the field of functional health drinks.

### BACKGROUND TECHNIQUE

Maca (Lepidium meyenii Walp) is a plant of the Lepidium genus, crucifer family native to the Andes region with an altitude of 3500-4500 m in South America. Maca has been eaten as food in South America for 5800 years, and used traditionally as food and herbal medicine for improving sexual function, enhancing fertility, ameliorating fatigue and tumor, and treating depression, asthma and menopausal syndrome. Maca has also been planted in Tashkurgan County in Xinjiang Region and Lijiang area in Yunnan province in China. Maca powder was approved as New resource food on Announcement No. 13 issued by Ministry of Health of China in 2011.

Maca is rich in nutrients. The dried maca root contains 10.2% proteins, 9-12% sugars, 59% carbohydrates, and a remarkable amount of minerals such as potassium, calcium, iron, zinc, etc. The maca has an appropriate amino acid constitution which has a high amount of branched amino acids which have important physiological activities. The maca comprises a less amount of fatty acids, up to 52% of which is unsaturated ones such as linoleic acid, linolenic acid, etc. The proteins, amino acids, carbohydrates, minerals and so on contained in maca have important contributions to anti-anemia, anti-blood-sugar-decline-after exercise, anti-fatigue activities of maca. Maca is also relatively rich in maca polysaccharides which are active substances capable of clearing superoxide radicals and inhibiting hydroxyl radicals.

Maca alkaloids are important secondary metabolites of the maca. Macamide and macaene, which are alkaloids of amides and contain special amide bond formed between amino group and fatty acid, are active ingredients obtained from maca extract by a Chinese American botanist B. L. Zheng in 1999. Macamide is an ingredient uniquely found in maca, and macaene, which is an acyclic ketoacid with small polarity, is normally found in chloroform extract of maca and also can be dissolved in methanol. Both the macamide and macaene play important roles in regulation of human endocrine system, such as systemically regulate body function, balance endocrine system, and improve sexual function, just like hormones. In 1980, glucosinolates (β-thioglucoside-N-hydroxysulfates) and isocyanates having volatility were found in maca by Johns. It has been found that isothiocyanates include mainly p-methoxybenzyl isothiocyanate and benzyl isothiocyanate. Glucosinolates are hydrophilic anionic secondary metabolites containing sulfur and nitrogen in maca which have a variable side chain (R) and a thio-β-D-glucopyranose group in molecules. The glucosinolates can produce cyanide, oxazolidine thione, thiocyanate and isothiocyanate enzymatically by myrosinase or bacterial enzymes in gastrointestinal tract. It was predicted that the fertility and sexual function-improving activity of the maca are possibly attributed to the glucosinolate and benzyl isothiocyanate which were also reported to have activity against cancers, such as stomach, esophagus, and lung cancer and leukemia and so on.

Fructus lycii is a dried ripe fruit of Lycium chinense Mill. or Lycium barbarum L. of Solanaceae family, and grown mainly in Hebei and Ningxia provinces in China, and also in Japan, Korea, Europe and North America, etc. Fructus lycii is an important herbal drugs in Chinese traditional medicine from ancient, and written as enhancing kidney, generating sperm, protecting liver, sparkling eyes, strengthening bones, ameliorating fatigue, lighting up face, calming nerve, and lengthening life in the “Compendium of Materia Medica”, as strengthening muscles and bones along with long-term usage in “Shen Nong's Herbal Classic”, as invigorating essential qi in “Mingyi Bielu”, and as invigorating body and ameliorating fatigue in “Herbal Food Medicine”. As verified by modern immunologic experiments and clinical medical experiments, Lycium barbarum L. has activities like immunomodulatory function-improvement, anti-oxidation, anti-aging, anti-fatigue, anti-cancer, anti-tumor, anti-hypertension, anti-hyperlipidemia, anti-hyperglycemia, protection of liver, kidney and neural system, and acceleration of excretion of harmful substances, especially heavy metals and carcinogens. Fructus lycii was recognized officially as both food and drug by Ministry of Health of China in 1988. Fructus lycii comprises mainly active ingredients, such as alkaloids, flavonoids and glycosides thereof, organic acids, coumarins, lignans, polysaccharides, etc.

Fructus rubi (i.e. Rubus chingii Hu) is a plant of Rubus genus, Rosaceae family, and grown mainly in East China, such as Zhejiang, Fujian, Anhui, Jiangxi, Jiangsu, etc. The immature dried fruit of the Rubus chingii Hu is used as a medicine for strengthen kidney, arresting spontaneous emission, reducing excessive urination, and for treating liver deficiency, enuresis, frequent micturition, impotence, premature ejaculation, spermatorrhea, spontaneous emission, etc. As verified by modern pharmacological research, Fructus rubi warms kidney, reinforces yang, acts against mutagenesis, oxidation, improves memory, delays aging, enhances immune activity, and inhibits bacteria. It was also observed from vaginal smear and endometrial slice of rats and rabbits that the fructus rubi administered had estrogen-like activities. The chemical components reported so far to be found in the fructus rubi comprises Fupenzic acid, Ellagic acid, β-sitosterol, Labdane diterpene glycoside goshonoside F5, and flavonoids triterpene acid.

Human gastrointestinal tract is colonized by a huge amount of microflora in which probiotics play crucial role on human health. Probiotics can catabolize food components which cannot be catabolized by digestive system, produce short-chain fatty acids, and supply nutrients for gastrointestinal mucosal cells. Probiotics can also convert inactive precursors of drug components to its active forms by catabolizing the precursors, especially naturally occurring ones, ingested orally. Currently used Probiotic formulations are mainly ones comprising simply live probiotics or ones comprising simply probiotic cells.

Currently used maca-containing products include maca liquor, maca jam, maca gum, etc. The more frequently seen maca-containing products are health foods and medicines made from powder of dried maca root or from concentrated maca extract. Dozens of health products mainly for enhancing energy, increasing fertility, improving sexual function and treating menopausal syndrome, etc. have been developed from worldwide including United States, Japan, Australia, Spain, Peru, United Kingdom, Taiwan, Hong Kong, etc. The commercially available maca-containing products are mainly made by extracting the maca or grinding the maca directly in combination with other active components, and there is no a product made by fermenting the maca or a composition containing the maca with probiotics yet.

### The Content of the Invention

The present invention provides a probiotic-fermented composition made of maca, fructus lycii and fructus rubi, more particularly a fermented composition obtained by enzymatically hydrolyzing an extract of maca, fructus lycii and fructus rubi, and fermenting the hydrolysate with multiple enteric probiotics, and a method for preparing the composition, as well as use of the composition in the manufacture of a health product, food or beverage for improving fatigue tolerance and sexual function.

One purpose of the present invention is to provide a probiotic-fermented maca composition for improving fatigue tolerance and sexual function, wherein the composition is obtained by

extracting the maca, fructus lycii and fructus rubi with water, hydrolyzing the extracts enzymatically, and subjecting the hydrolysate to a probiotic-fermentation; wherein said composition comprises: 1.0-10% (g/ml) of maca, 2.0-8.0% (g/ml) of fructus lycii, and 2.0-8.0% (g/ml) of fructus rubi, wherein the enzyme used in the enzymatic hydrolysis is a high temperature amylase with a high optimal temperature; and wherein the probiotics are Bifidobacterium and Lactobacillus.

Preferably, said composition of the present invention comprises: 3.0% (g/ml) of maca, 3.0% (g/ml) of fructus lycii, and 3.0% (g/ml) of fructus rubi.

Preferably, said Bifidobacterium used in the present invention is selected from Bifidobacterium bifidum, Bifidobacterium breve, Bifidobacterium adolescentis or Bifidobacterium infantis; and said Lactobacillus used in the present invention is selected from Lactobacillus acidophilus, Lactobacillus delbrueckii, Lactobacillus casei, Lactobacillus rhamnosus or Lactobacillus plantarum. The strains are obtained from China General Microbiological Culture Collection Center (CGMCC China, Beijing) or isolated from feces or entero-mucosal membrane tissue of a healthy human.

To improve the taste of the composition, it is further preferable to add sweeteners into the composition of the present invention, wherein sweeteners are sucrose which is present in an amount of 3.0% (g/ml), and sucralose which is present in an amount of 0.008% (g/ml) respectively.

Another purpose of the present invention is to provide a method for preparing a probiotic-fermented maca composition for improving fatigue tolerance and sexual function, comprising the steps of:

(1) obtaining a mixed powder of maca, fructus lycii and fructus rubi by grinding the maca to a powder with a fineness of 100 mesh or more, grinding the mixture of the fructus lycii and fructus rubi to a powder with a fineness of 10 mesh or more, and mixing the above-obtained powders together;

maca, fructus lycii and fructus rubi are ground by conventional means resulting increased specific surface areas which facilitate dilution of active ingredients and thus increase yield of extraction.

(2) obtaining a complex extract of the maca, fructus lycii and fructus rubi by immersing the mixed powder obtained in the above step (1) in water weighed 10-16 times greater than the mixed powder for 30 min to obtain a mixture, extracting the mixture at around 115° C. under high-pressure for 40 minutes to obtain an extract, hydrolyzing the extract with a high-temperature amylase after cooling to 85-90° C. and adjusting the pH to around 5.5, filtering and concentrating the hydrolysate under reduced pressure after sterilization to obtain a filtrate from which insoluble residues have been removed, and adding into the filtrate an amount of glucose and inorganic salts which is followed by adjustment of pH to 6.5-7.0 and sterilization;

(3) obtaining a probiotic-fermented maca composition by a probiotic-fermentation which comprises: seeding 0.5% (v/v) of one or two or more pre-cultured Bifidobacterium into the complex extract obtained in the above step (2) when it is cooled to about 39° C. and culturing for about 3 to 8 hours, seeding thereto further 0.5% (v/v) of one or two or more pre-cultured Lactobacillus and culturing for about 20 to 22 hours, decreasing the culture temperature to 28° C. when the pH is dropped to 4.2 or less, and continuing the fermentation until the pH is dropped to 4.0 and stabilizing the resultant ferment.

Preferably, in the method of present invention as mentioned above, said composition comprises: 1.0-10% (g/ml) of maca, 2.0-8.0% (g/ml) of fructus lycii, and 2.0-8.0% (g/ml) of fructus rubi,

Preferably, in the method of present invention as mentioned above, the ratio between the high-temperature amylase and the mixed powder in the step (2) is 30-40 U/g; the glucose is added into the filtrate in an amount of 0.5% (g/ml); and the inorganic salts are 5.0 mg/100 ml of FeSO₄.7H₂O, 4.4 mg/100 ml of ZnSO₄.7H₂O, and 50 mg/100 ml of MgSO₄.7H₂O.

Preferably, in the method of present invention as mentioned above, to improve the taste of the composition, the method further comprises, after the enzymatic hydrolysis and filtration in the step (2), adding sweeteners which are 3.0 g/100 ml of sucrose, and 0.008 g/100 ml of sucralose.

Preferably, in the method of present invention as mentioned above, said Bifidobacterium is selected from Bifidobacterium bifidum, Bifidobacterium breve, Bifidobacterium adolescentis or Bifidobacterium infantis; and said Lactobacillus is selected from Lactobacillus acidophilus, Lactobacillus delbrueckii, Lactobacillus casei, Lactobacillus rhamnosus or Lactobacillus plantarum.

In a preferred embodiment, the composition of the present invention has a total sugar-content of 20-100 mg/ml and has a total probiotics quantity of 2.0-8.0×10⁸ cells/ml as counted under microscope using blood cell counter (see Preparation Example 1).

A further purpose of the present invention is to provide use of the composition according to the present invention in the manufacture of a health product, food or beverage for improving fatigue tolerance and sexual function in a subject.

The composition of the present invention can be formulated directly into food or health product suitable for use in a subject suffered from fatigue and sexual dysfunction.

In a preferred embodiment, the composition prepared by the method of the present invention can be used to prepare a health product or a beverage for improving fatigue tolerance and sexual function.

For example, additional natural or synthetic ingredient(s) having the same, similar or different biological activities and having facilitative or synergistic activity with the active ingredients of the present invention while having no antagonistic activity with each other can be added into the composition of the present invention or supernatant thereof to form a mist, emulsion and the like suitable for oral administration by conventional means known in the art.

The composition of the present invention can also be dried and formulated into a solid powder into which additional natural or synthetic ingredient(s) having the same, similar or different biological activities and having facilitative or synergistic activity with the active ingredients of the present invention while having no antagonistic activity with each other can be added to form a capsule, tablet, granule and the like suitable for oral administration by conventional means known in the art.

To test anti-fatigue and sexual function-improving activity of the composition of the present invention, the effects of the composition of the present invention on duration time of pole-climbing and swimming, and levels of serum urea nitrogen, blood lactate and liver glycogen in mice were investigated. The result showed that the composition of the present invention could remarkably prolong the duration time of pole-climbing and swimming, keep the levels of serum urea nitrogen and blood lactate remarkably below the control, and keep the level of liver glycogen above the control in mice which indicated that the composition of the present invention had an ability to significantly improve fatigue-tolerance in a mouse. At the same time, the effects of the composition of the present invention on mating latency and mating frequency in male mice were investigated. The result showed that the composition of the present invention can significantly reduce the mating latency and increase mating frequency in male mice which indicated that the composition of the present invention had an ability to significantly improve sexual function in mice. The experiments also compared effects of the compositions fermented or unfermented with probiotics on fatigue-tolerance and sexual function improvement in mice and proved that the fermented composition was superior to the unfermented one which indicated that the method of the present invention improved physiological activity of the composition made of mixture of maca, fructus lycii and fructus rubi.

### EXAMPLES

### Preparation Example 1: Preparation of a Probiotic-Fermented Composition A

30 g of dried maca (Lepidium meyenii Walp), which had been ground and sieved through a sieve of 100 mesh, and 30 g of dried fructus lycii (Lycium barbarum L) and 30 g of dried fructus rubi (Rubus chingii Hu), which had been mixed, ground and sieved through a sieve of 10 mesh, were mixed together to obtain a mixed powder of maca, fructus lycii and fructus rubi. The mixed powder was immersed in water weighed 12 times greater than the mixed powder for 30 minutes to obtain a mixture. The mixture was extracted at around 115° C. under high-pressure for 40 minutes to obtain an extract. After the extract had been cooled to 85-90° C. and the pH of the extract had been adjusted to around 5.5 with sodium hydroxide, a 2700 units (30 U/g mixed powder) of high-temperature amylase which had been diluted with water was added into the extract and stirred well, and the enzymolysis was allowed to occur with the temperature being maintained at 85-90° C. The enzymolysis was allowed to end when the indicator of iodine was not colored blue remarkably as compared to the control which was not hydrolyzed enzymatically. The enzyme was inactivated at 100° C. for 10 minutes, and the hydrolysate was filtered though a filter of 200 mesh, centrifuged and recovered for the filtrate. The filtrate was concentrated to a concentration of 9.0% (g/ml) under reduced pressure. 0.5% (g/ml) of glucose and inorganic salts (5.0 mg/100 ml of FeSO₄.7H₂O, 4.4 mg/100 ml of ZnSO₄.7H₂O, and 50 mg/100 ml of MgSO₄.7H₂O), 3.0 g/100 ml of sucrose, and 0.008 g/100 ml of sucralose were added into the concentrated filtrate which was followed by adjustment of pH to about 7.0 and sterilization at 115° C. for 40 minutes to obtain a complex extract of the maca, fructus lycii and fructus rubi. A 0.5% (v/v) of pre-cultured Bifidobacterium bifidum (isolated from normal human body and identified by the Chinese Academy of Sciences Institute of Microbiology) and Bifidobacterium breve (AS 1.2213) were seeded into the complex extract when it had been cooled to about 39° C., and allowed to ferment for 6 hours. A 0.5% (v/v) of pre-cultured Lactobacillus delbrueckii (AS 1.1480) and Lactobacillus plantarum (AS 1.19) were further seeded thereto, and allowed to ferment at 37° C. for 20-22 hours. The temperature of fermentation was decreased when the pH was dropped to 4.2 or less and the fermentation was continued until the pH was dropped to 4.0. The ferment was stabilized in a water bath of 60° C. for 4 hours after the fermentation was stopped to obtain the Composition A which had a total probiotics quantity of 3.2×10⁸ cells/ml as counted under microscope using blood cell counter and had a total sugar-content of 35 mg/ml.

### Preparation Example 2: Preparation of a Probiotic-Fermented Composition B

50 g of dried maca (Lepidium meyenii Walp), which had been ground and sieved through a sieve of 100 mesh, and 40 g of dried fructus lycii (Lycium barbarum L) and 20 g of dried fructus rubi (Rubus chingii Hu), which had been mixed, ground and sieved through a sieve of 10 mesh, were mixed together to obtain a mixed powder of maca, fructus lycii and fructus rubi. The mixed powder was immersed in water weighed 14 times greater than the mixed powder for 30 minutes to obtain a mixture. The mixture was extracted at around 115° C. under high-pressure for 40 minutes to obtain an extract. After the extract had been cooled to 85-90° C. and the pH of the extract had been adjusted to around 5.5 with sodium hydroxide, a 3850 units (35 U/g mixed powder) of high-temperature amylase which had been diluted with water was added into the extract and stirred well, and the enzymolysis was allowed to occur with the temperature being maintained at 85-90° C. The enzymolysis was allowed to end when the indicator of iodine was not colored blue remarkably as compared to the control which was not hydrolyzed enzymatically. The enzyme was inactivated at 100° C. for 10 minutes, and the hydrolysate was filtered though a filter of 200 mesh, centrifuged and recovered for the filtrate. The filtrate was concentrated to a concentration of 11.0% (g/ml) under reduced pressure. 0.5% (g/ml) of glucose and inorganic salts (5.0 mg/100 ml of FeSO₄.7H₂O, 4.4 mg/100 ml of ZnSO₄.7H₂O, and 50 mg/100 ml of MgSO₄.7H₂O), 3.0 g/100 ml of sucrose, and 0.008 g/100 ml of sucralose were added into the concentrated filtrate which was followed by adjustment of pH to about 7.0 and sterilization at 115° C. for 40 minutes to obtain a complex extract of the maca, fructus lycii and fructus rubi. A 0.5% (v/v) of pre-cultured Bifidobacterium bifidum (isolated from normal human body and identified by the Chinese Academy of Sciences Institute of Microbiology) and Bifidobacterium adolescentis (AS1.2190) were seeded into the complex extract when it had been cooled to about 39° C., and allowed to ferment for 6 hours. A 0.5% (v/v) of pre-cultured Lactobacillus acidophilus (AS 1.1854) and Lactobacillus plantarum (AS 1.19) were further seeded thereto, and allowed to ferment at 37° C. for 20-22 hours. The temperature of fermentation was decreased when the pH was dropped to 4.2 or less and the fermentation was continued until the pH was dropped to 4.0. The ferment was stabilized in a water bath of 60° C. for 4 hours after the fermentation was stopped to obtain the Composition B which had a total probiotics quantity of 4.2×10⁸ cells/ml as counted under microscope using blood cell counter and had a total sugar-content of 65 mg/ml.

### Preparation Example 3: Preparation of a Probiotic-Fermented Composition C

80 g of dried maca (Lepidium meyenii Walp), which had been ground and sieved through a sieve of 100 mesh, and 50 g of dried fructus lycii (Lycium barbarum L) and 20 g of dried fructus rubi (Rubus chingii Hu), which had been mixed, ground and sieved through a sieve of 10 mesh, were mixed together to obtain a mixed powder of maca, fructus lycii and fructus rubi. The mixed powder was immersed in water weighed 16 times greater than the mixed powder for 30 minutes to obtain a mixture. The mixture was extracted at around 115° C. under high-pressure for 40 minutes to obtain an extract. After the extract had been cooled to 85-90° C. and the pH of the extract had been adjusted to around 5.5 with sodium hydroxide, a 6000 units (40 U/g mixed powder) of high-temperature amylase which had been diluted with water was added into the extract and stirred well, and the enzymolysis was allowed to occur with the temperature being maintained at 85-90° C. The enzymolysis was allowed to end when the indicator of iodine was not colored blue remarkably as compared to the control which was not hydrolyzed enzymatically. The enzyme was inactivated at 100° C. for 10 minutes, and the hydrolysate was filtered though a filter of 200 mesh, centrifuged and recovered for the filtrate. The filtrate was concentrated to a concentration of 15.0% (g/ml) under reduced pressure. 0.5% (g/ml) of glucose and inorganic salts (5.0 mg/100 ml of FeSO₄.7H₂O, 4.4 mg/100 ml of ZnSO₄.7H₂O, and 50 mg/100 ml of MgSO₄.7H₂O), 3.0 g/100 ml of sucrose, and 0.008 g/100 ml of sucralose were added into the concentrated filtrate which was followed by adjustment of pH to about 7.0 and sterilization at 115° C. for 40 minutes to obtain a complex extract of the maca, fructus lycii and fructus rubi. A 0.5% (v/v) of pre-cultured Bifidobacterium infantis (AS1.853) and Bifidobacterium breve (AS 1.2213) were seeded into the complex extract when it had been cooled to about 39° C., and allowed to ferment for 6 hours. A 0.5% (v/v) of pre-cultured Lactobacillus delbrueckii (AS 1.1480) and Lactobacillus rhamnosus (AS 1.22) were further seeded thereto, and allowed to ferment at 37° C. for 20-22 hours. The temperature of fermentation was decreased when the pH was dropped to 4.2 or less and the fermentation was continued until the pH was dropped to 4.0. The ferment was stabilized in a water bath of 60° C. for 4 hours after the fermentation was stopped to obtain the Composition C which had a total probiotics quantity of 5.8×10⁸ cells/ml as counted under microscope using blood cell counter and had a total sugar-content of 84 mg/ml.

### Experimental Example 1: Investigation of Effect of the Fermented Composition of the Present Invention on Fatigue-Tolerance

The present experimental example aimed to investigate effect of the composition comprising maca, fructus lycii and fructus rubi fermented by several probiotics on fatigue-tolerance in mice by pole-climbing test, loaded swimming test, blood lactate measurement, serum urea nitrogen measurement, and liver glycogen measurement.

The Compositions A, B and C obtained in the Preparation Examples 1, 2 and 3 were served as “fermented samples”, while the compositions of the same components without subjecting to fermentation with Bifidobacterium and Lactobacillus were served as “unfermented samples”.

Experimental animals: in this experiment, 1-month-old healthy SPF male Kunming mice with body-weight of 20±3 g provided by Research Center for Experimental Animal in Shandong University of Traditional Chinese Medicine were used and randomized.

Doses: The recommended dose for human was 100 mL/day/60 kg body weight. The equivalent dose for mouse was 10 times more than that of human. An oral dosage form of 16.7 mL/day/kg body weight which is mouse dose equivalent to recommended human dose was set as middle dose, while the 8.3 mL/Day/kg body weight which is 0.5 times of the middle dose and 33.3 mL/day/kg body weight which is 2 times of the middle dose were set as low dose and high dose respectively. The doses were diluted with distilled water and administered to the mice intragastrically at 1.2 mL/day for 25 days continuously.

Instrument: a pole-climbing shelf, a swimming tank, UV spectrophotometer 754, ES-200 electronic balance, centrifuge, a swimming tank, intragastric feeder and so on.

Reagents: A serum urea nitrogen detection Kit (diacetyl oxime method).

5% of Trichloroacetic acid (TCA), glucose standard solution, concentrated sulfuric acid, anthrone reagent, 4% copper sulfate solution, 1% sodium fluoride solution, protein precipitant, 1.5% para-hydroxybiphenyl solution, the standard solution of lactic acid.

### Experimental Method

### 1. Pole-Climbing Test:

The mice were placed on a rod of plexiglass in a pole-climbing shelf with their muscles being on a state of static tension, 30 min after the last administration of the test substance. The duration time of the mice retaining on the rod before they falling down due to fatigue was measured. The test was stopped upon the 3^{rd} falling, and the mean of the durations tested 3 times was set as “pole-climbing duration”.

### 2. Loaded Swimming Test:

The mice were loaded with lead wires of 5% of their body-weight, 30 min after the last administration of the test substance. The mice were placed in a swimming tank with a water depth of 30 cm or more and a water temperature of 25° C., and allowed to swim. The duration from beginning to swim to entire head being immersed in the water while being unable to float over the surface of the water for 8 seconds was set as “swimming duration”.

### 3. Measurement of Serum Urea Nitrogen:

The mice were placed in water at a temperature of 25° C. and allowed to swim for 90 min, 30 min after the last administration of the test substance. Serum was taken from blood sampled from eyes removed from the mice, and tested with a kit according to the procedure as shown in the Table 1.

TABLE 1

| Reagents | Blank | Standard | Test |
|---|---|---|---|
| BUN20 mg/dL Standard | | 0.02 | |
| Solution (mL) | | | |
| Sample (mL) | | | 0.02 |
| Diacetyl oxime Solution | 3.0 | 3.0 | 3.0 |
| (mL) | | | |
| Ferric chloride-phosphate | 2.5 | 2.5 | 2.5 |
| Solution (mL) | | | |

The serum were mixed with the agents thoroughly, placed in a boiling water bath for 10 minutes, and then cooled in cold water. Absorbances at 520 nm, i.e. A_{standard}, A_{Sample}, and A_{Blank}, were measured and calibrated with those measured with distilled water.

### 4. Measurement of Blood Lactate

The mice were placed in water at a temperature of 25° C. and allowed to swim for 60 min, 30 min after the last administration of the test substance. Blood was sampled from eyes removed from the mice after leaving arrested for 15 min. 0.48 mL of 1% NaF solution was added into a 5 ml centrifuge tube, into bottom of which 20 μL of whole blood was then accurately added with the pipette being washed with the supernatant by multiple-pipetting. 1.5 mL protein precipitant was further added into the tube, and the tube was vibrated thoroughly and centrifuged at 3000 r/min for 10 min, and the supernatant was subjected to operation in accordance with Table 2.

TABLE 2

| | Blank Tube | Standard Tube | Test Tube |
|---|---|---|---|
| | (mL) | (mL) | (mL) |
| Precipitating Agent-NaF | 0.5 | — | — |
| Mix-Solution | | | |
| Lactic acid Standard Solution | — | 0.5 | — |
| Sample | — | — | 0.5 |
| 4%CuSO₄ | 0.1 | 0.1 | 0.1 |
| Concentrated sulfuric acid | 3 | 3 | 3 |
| Mixed thoroughly, placed in | | | |
| a boiling water bath for | | | |
| 5 min, and cooled in icy water | | | |
| for 10 min | | | |
| 1.5% p-Hydroxydiphenyl | 0.1 | 0.1 | 0.1 |

The resultant was shaken thoroughly and placed in a 30° C. water bath for 30 min with shaking at every 10 min after completion of the foregoing steps. The resultant was then placed in a boiling water bath for 90 s, cooled to room-temperature, and then subjected to colorimetry at 560 nm and calibrated with blank tube.

5. Measurement of Liver Glycogen with Anthrone Method

The mice were sacrificed, 30 min after the last administration of the test substance. Livers were taken from the mice, rinsed with a saline, and dried with a filter paper. An accurately weighed 200 mg of liver piece was placed in a tube into which 4 mL of TCA was added, and then homogenized for 1 min. The homogenate was poured into a centrifuge tube, and centrifuged at 3000 r/min for 15 min, and the supernatant was transferred into another tube. 4 mL of TCA was added to the precipitating agent, homogenized for 1 min, and further centrifuged for 15 min, and the supernatant was pooled with the previous supernatant and mixed thoroughly. 1 mL of the pooled supernatant was placed into 10 ml centrifuge tubes into which 4 mL of 95% ethanol was added, and mixed thoroughly until there was no interface between the two liquids. The tube was plugged with a clean plug, and left at room temperature overnight. The tube was centrifuged at 3000 r/min for 15 min after precipitation was completed. The supernatant was discarded carefully and the tube was left upside-down for 10 min. The glycogen was dissolved in 2 mL distilled water with the glycogen on the wall of the tube being washed down by the distilled water, and shaken for thorough dissolution. 0.1 ml of the solution was diluted in 0.9 mL of distilled water.

Blank Agent: 1 mL of distilled water was placed in a clean centrifuge tube and set as the Blank Agent.

Standard tube: 0.1 mL of standard solution containing 100 mg/dL glucose was placed into a tube into which 1.5 ml of distilled water was added.

Assay: 5 mL of anthrone reagent was injected directly into center of each tube which is in a cold water bath, and mixed thoroughly. Upon the temperature of the mixture was decreased to that of the cold water bath, the tube was transferred to a boiling water bath for 15 min. The tube was then transferred to an icy water bath until it cooled to room-temperature. The content of the tube was transferred to a colorimetric tube, and absorbance at 620 nm was measured and calibrated with that measured with the Blank tube. The absorbance of the standard tube was also measured. The measured absorbances were converted to content of liver glycogen against weight of the liver (expressed at g/100 g of liver).

### Results

1. Pole-climbing Test with Mice

TABLE 3
Effects of the fermented Compositions A, B and C
and the corresponding compositions without fermentation
on pole-climbing durations in mice (X ± SD)

| | | Number | |
|---|---|---|---|
| | | of | Pole-Climbing |
| Groups | | Animals | Durations (sec) |
| | 1 Control | 10 | 370.00 ± 37.82 |
| Fermented | A₁ Unfermented | 10 | 443.10 ± 49.49* |
| and | High-Dose Group | | |
| unfermented | A₂ Unfermented | 10 | 442.70 ± 59.04* |
| Composition | Middle-Dose Group | | |
| A | A₃ Unfermented | 10 | 375.50 ± 70.83 |
| | Low-Dose Group | | |
| | A₄ Fermented | 10 | 511.60 ± 50.92*^{▴} |
| | High-Dose Group | | |
| | A₅ Fermented | 10 | 502.40 ± 35.16*^{▴} |
| | Middle-Dose Group | | |
| | A₆ Fermented | 10 | 486.40 ± 67.20*^{▴} |
| | Low-Dose Group | | |
| Fermented | B₁ Unfermented | 10 | 472.10 ± 32.26* |
| and | High-Dose Group | | |
| unfermented | B₂ Unfermented | 10 | 455.50 ± 46.30* |
| Composition | Middle-Dose Group | | |
| B | B₃ Unfermented | 10 | 378.60 ± 63.21 |
| | Low-Dose Group | | |
| | B₄ Fermented | 10 | 564.20 ± 54.31*^{▴} |
| | High-Dose Group | | |
| | B₅ Fermented | 10 | 546.30 ± 36.64*^{▴} |
| | Middle-Dose Group | | |
| | B₆ Fermented | 10 | 528.40 ± 48.02*^{▴} |
| | Low-Dose Group | | |
| Fermented | C₁ Unfermented | 10 | 502.30 ± 42.16* |
| and | High-Dose Group | | |
| unfermented | C₂ Unfermented | 10 | 494.50 ± 38.45* |
| Composition | Middle-Dose Group | | |
| C | C₃ Unfermented | 10 | 437.60 ± 32.18* |
| | Low-Dose Group | | |
| | C₄ Fermented | 10 | 598.30 ± 31.06*^{▴} |
| | High-Dose Group | | |
| | C₅ Fermented | 10 | 576.40 ± 42.28*^{▴} |
| | Middle-Dose Group | | |
| | C₆ Fermented | 10 | 554.20 ± 50.83*^{▴} |
| | Low-Dose Group | | |

*Significant difference compared with the control group (P < 0.05).
^{▴}Significant difference between the fermented and unfermented compositions at the same concentrations (P < 0.05).

The results (Table 3) showed that the pole-climbing durations of mice were increased by all of the fermented Composition A, B, and C at all doses, and the unfermented Compositions A and B at high and middle doses, and the unfermented Composition C at high, middle and low doses, with the fermented Compositions A, B and C at all doses acting significantly superior to the corresponding compositions at the corresponding doses without fermentation, which indicated that the extracts of maca, fructus lycii and fructus rubi fermented by probiotics were able to increase the pole-climbing duration of mice.

2. Swimming Test with Mice

TABLE 4
Effects of the fermented Compositions A, B and
C and the corresponding compositions without fermentation
on swimming duration in mice (X ± SD)

| | | Number | |
|---|---|---|---|
| | | of | Swimming |
| Groups | | Animals | durations (sec) |
| | Control | 10 | 362.31 ± 69.87 |
| Fermented | A₁ Unfermented | 10 | 426.40 ± 62.24* |
| and | High-Dose Group | | |
| unfermented | A₂ Unfermented | 10 | 401.18 ± 56.54 |
| Composition | Middle-Dose Group | | |
| A | A₃ Unfermented | 10 | 412.00 ± 44.19 |
| | Low-Dose Group | | |
| | A₄ Fermented | 10 | 503.40 ± 61.10*^{▴} |
| | High-Dose Group | | |
| | A₅ Fermented | 10 | 491.60 ± 57.31*^{▴} |
| | Middle-Dose Group | | |
| | A₆ Fermented | 10 | 483.10 ± 84.76*^{▴} |
| | Low-Dose Group | | |
| Fermented | B₁ Unfermented | 10 | 462.60 ± 53.12* |
| and | High-Dose Group | | |
| unfermented | B₂ Unfermented | 10 | 446.20 ± 48.62* |
| Composition | Middle-Dose Group | | |
| B | B₃ Unfermented | 10 | 408.54 ± 39.76 |
| | Low-Dose Group | | |
| | B₄ Fermented | 10 | 561.30 ± 62.14*^{▴} |
| | High-Dose Group | | |
| | B₅ Fermented | 10 | 547.60 ± 45.24*^{▴} |
| | Middle-Dose Group | | |
| | B₆ Fermented | 10 | 506.82 ± 54.68*^{▴} |
| | Low-Dose Group | | |
| Fermented | C₁ Unfermented | 10 | 503.62 ± 48.21* |
| and | High-Dose Group | | |
| unfermented | C₂ Unfermented | 10 | 485.25 ± 52.13* |
| Composition | Middle-Dose Group | | |
| C | C₃ Unfermented | 10 | 446.30 ± 32.18* |
| | Low-Dose Group | | |
| | C₄ Fermented | 10 | 601.20 ± 59.35*^{▴} |
| | High-Dose Group | | |
| | C₅ Fermented | 10 | 586.75 ± 46.12*^{▴} |
| | Middle-Dose Group | | |
| | C₆ Fermented | 10 | 543.50 ± 61.20*^{▴} |
| | Low-Dose Group | | |

*Significant difference compared with the control group (P < 0.05).
^{▴}Significant difference between the fermented and unfermented compositions at the same concentrations (P < 0.05).

The results (Table 4) show that the swimming durations of mice were increased by all of the fermented Compositions A, B and C at all doses and the unfermented Composition A at high dose, the unfermented Composition B at high dose, the unfermented Composition C at high, middle and low doses, with the fermented Compositions A, B and C at all doses acting significantly superior to the corresponding compositions at the corresponding doses without fermentation, which indicated that the extracts of maca, fructus lycii and fructus rubi fermented by probiotics were able to increase swimming duration in mice.

### 3. Level of Serum Urea Nitrogen in Mice

TABLE 5
Effect of fermented Compositions A, B and C and the
corresponding compositions without fermentation on
level of serum urea nitrogen in mice (X ± SD)

| | | Number | |
|---|---|---|---|
| | | of | Level of serum |
| Groups | | Animals | urea nitrogen |
| | Control | 10 | 31.29 ± 2.94 |
| Fermented | A₁ Unfermented | 10 | 28.81 ± 1.92 |
| and | High-Dose Group | | |
| unfermented | A₂ Unfermented | 10 | 29.07 ± 1.78 |
| Composition | Middle-Dose Group | | |
| A | A₃ Unfermented | 10 | 28.59 ± 2.28 |
| | Low-Dose Group | | |
| | A₄ Fermented | 10 | 26.97 ± 1.59* |
| | High-Dose Group | | |
| | A₅ Fermented | 10 | 27.31 ± 1.44* |
| | Middle-Dose Group | | |
| | A₆ Fermented | 10 | 27.97 ± 2.63 |
| | Low-Dose Group | | |
| Fermented | B₁ Unfermented | 10 | 28.35 ± 1.65 |
| and | High-Dose Group | | |
| unfermented | B₂ Unfermented | 10 | 28.98 ± 1.38 |
| Composition | Middle-Dose Group | | |
| B | B₃ Unfermented | 10 | 29.12 ± 2.06 |
| | Low-Dose Group | | |
| | B₄ Fermented | 10 | 26.34 ± 1.24* |
| | High-Dose Group | | |
| | B₅ Fermented | 10 | 26.83 ± 1.55* |
| | Middle-Dose Group | | |
| | B₆ Fermented | 10 | 27.25 ± 1.38* |
| | Low-Dose Group | | |
| Fermented | C₁ Unfermented | 10 | 27.13 ± 1.23* |
| and | High-Dose Group | | |
| unfermented | C₂ Unfermented | 10 | 27.64 ± 1.45* |
| Composition | Middle-Dose Group | | |
| C | C₃ Unfermented | 10 | 28.26 ± 1.63 |
| | Low-Dose Group | | |
| | C₄ Fermented | 10 | 25.08 ± 1.26* |
| | High-Dose Group | | |
| | C₅ Fermented | 10 | 26.42 ± 1.32* |
| | Middle-Dose Group | | |
| | C₆ Fermented | 10 | 26.89 ± 1.48* |
| | Low-Dose Group | | |

*Significant difference compared with the control group (P < 0.05).
▴ Significant difference between the fermented and unfermented compositions at the same concentrations (P < 0.05).

The results (Table 5) show that the level of serum urea nitrogen in mice was decreased by all of the fermented Composition A at high dose, the fermented Compositions B and C at high, middle and low doses and the unfermented Composition C at high dose.

### 4. Level of Liver Glycogen in Mice

TABLE 6
Effects of the fermented Compositions A, B and C and
the corresponding compositions without fermentation
on the level of liver glycogen in mice (X ± SD)

| | | Number | Level of |
|---|---|---|---|
| | | of | Liver |
| Groups | | Animals | Glycogen |
| | Control | 10 | 2.21 ± 0.24 |
| Fermented | A₁ Unfermented | 10 | 2.41 ± 0.30 |
| and | High-Dose Group | | |
| unfermented | A₂ Unfermented | 10 | 2.21 ± 0.17 |
| Composition | Middle-Dose Group | | |
| A | A₃ Unfermented | 10 | 2.19 ± 0.16 |
| | Low-Dose Group | | |
| | A₄ Fermented | 10 | 2.79 ± 0.34* |
| | High-Dose Group | | |
| | A₅ Fermented | 10 | 2.47 ± 0.37 |
| | Middle-Dose Group | | |
| | A₆ Fermented | 10 | 2.42 ± 0.24 |
| | Low-Dose Group | | |
| Fermented | B₁ Unfermented | 10 | 2.69 ± 0.21* |
| and | High-Dose Group | | |
| unfermented | B₂ Unfermented | 10 | 2.31 ± 0.15 |
| Composition | Middle-Dose Group | | |
| B | B₃ Unfermented | 10 | 2.27 ± 0.16 |
| | Low-Dose Group | | |
| | B₄ Fermented | 10 | 3.16 ± 0.35*^{▴} |
| | High-Dose Group | | |
| | B₅ Fermented | 10 | 2.89 ± 0.23*^{▴} |
| | Middle-Dose Group | | |
| | B₆ Fermented | 10 | 2.52 ± 0.14 |
| | Low-Dose Group | | |
| Fermented | C₁ Unfermented | 10 | 2.93 ± 0.32* |
| and | High-Dose Group | | |
| unfermented | C₂ Unfermented | 10 | 2.79 ± 0.25* |
| Composition | Middle-Dose Group | | |
| C | C₃ Unfermented | 10 | 2.71 ± 0.15* |
| | Low-Dose Group | | |
| | C₄ Fermented | 10 | 3.52 ± 0.19*^{▴} |
| | High-Dose Group | | |
| | C₅ Fermented | 10 | 3.36 ± 0.17*^{▴} |
| | Middle-Dose Group | | |
| | C₆ Fermented | 10 | 3.18 ± 0.21*^{▴} |
| | Low-Dose Group | | |

*Significant difference compared with the control group (P < 0.05).
^{▴}Significant difference between the fermented and unfermented compositions at the same concentrations (P < 0.05).

The results (Table 6) show that the levels of liver glycogen in mice were increased by all of the fermented Composition A at high dose, the fermented Composition B at high dose, the fermented Composition C at high, middle and low doses and the unfermented Composition B at high dose, the unfermented Composition C at high, middle and low doses, with the fermented Composition B at high and middle doses and the fermented Composition C at all doses acting significantly superior to the corresponding compositions at the corresponding doses without fermentation.

### 5. Level of Blood Lactate

TABLE 7
Effects of the fermented Compositions A, B and C
and the corresponding compositions without fermentation
on level of blood lactate in mice (X ± SD)

| | | Number | |
|---|---|---|---|
| | | of | Level of blood |
| Groups | | Animals | lactate |
| | Control | 10 | 33.89 ± 3.98 |
| Fermented | A₁ Unfermented | 10 | 22.70 ± 2.95* |
| and | High-Dose Group | | |
| unfermented | A₂ Unfermented | 10 | 27.87 ± 3.22* |
| Composition | Middle-Dose Group | | |
| A | A₃ Unfermented | 10 | 27.20 ± 3.76* |
| | Low-Dose Group | | |
| | A₄ Fermented | 10 | 20.47 ± 2.17* |
| | High-Dose Group | | |
| | A₅ Fermented | 10 | 22.16 ± 2.44*^{▴} |
| | Middle-Dose Group | | |
| | A₆ Fermented | 10 | 24.33 ± 4.28* |
| | Low-Dose Group | | |
| Fermented | B₁ Unfermented | 10 | 20.65 ± 2.81* |
| and | High-Dose Group | | |
| unfermented | B₂ Unfermented | 10 | 25.36 ± 2.65* |
| Composition | Middle-Dose Group | | |
| B | B₃ Unfermented | 10 | 26.05 ± 2.13* |
| | Low-Dose Group | | |
| | B₄ Fermented | 10 | 18.54 ± 3.15* |
| | High-Dose Group | | |
| | B₅ Fermented | 10 | 19.35 ± 2.86*^{▴} |
| | Middle-Dose Group | | |
| | B₆ Fermented | 10 | 20.48 ± 2.15*^{▴} |
| | Low-Dose Group | | |
| Fermented | C₁ Unfermented | 10 | 19.65 ± 2.98* |
| and | High-Dose Group | | |
| unfermented | C₂ Unfermented | 10 | 20.58 ± 3.16* |
| Composition | Middle-Dose Group | | |
| C | C₃ Unfermented | 10 | 22.15 ± 2.65* |
| | Low-Dose Group | | |
| | C₄ Fermented | 10 | 14.24 ± 2.46*^{▴} |
| | High-Dose Group | | |
| | C₅ Fermented | 10 | 15.68 ± 2.14* |
| | Middle-Dose Group | | |
| | C₆ Fermented | 10 | 15.95 ± 3.02*^{▴} |
| | Low-Dose Group | | |

*Significant difference compared with the control group (P < 0.05).
^{▴}Significant difference between the fermented and unfermented compositions at the same concentrations (P < 0.05).

The results (Table 7) show that the levels of blood lactate were decreased by the fermented Compositions A, B and C and the unfermented Compositions A, B and C at all doses in mice after exercise, with the fermented Composition A at middle dose, the fermented Composition B at middle and low doses, the fermented Composition C at high and low doses acting superior to the corresponding compositions at the corresponding doses without fermentation.

### Conclusions

The results of pole-Climbing test, swimming test, and measurement of levels of the serum urea nitrogen, liver glycogen, and blood lactate in mice showed that the compositions made of maca, fructus lycii and fructus rubi fermented by probiotics were able to ameliorate fatigue, and the probiotic-fermented compositions acted significantly superior to the corresponding compositions without fermentation, which indicated that the fermentation with probiotics was able to increase the anti-fatigue activities of the compositions made of maca, fructus lycii and fructus rubi.

### Experimental Example 2: Investigation of Effects of the Fermented Composition of the Present Invention on Improvement of Sexual Function

The present experimental example aimed to investigate effects of the composition comprising maca, fructus lycii and fructus rubi fermented by several probiotics on sexual function in mice by measuring mating latency and mating frequency within 20 min in male mice.

The fermented Compositions A, B and C obtained in the Preparation Examples were served as “fermented samples”, while the compositions of the same components without subjecting to fermentation were served as “unfermented samples”.

Experimental animals: in this experiment, 1-month-old healthy SPF Kunming mice (male:female=1:3) with body-weight of 20±3 g provided by Research Center for Experimental Animal in Shandong University of Traditional Chinese Medicine were used and randomized.

Doses: The recommended dose for human was 100 mL/day/60 kg body weight. The equivalent dose for mouse was 10 times more than that of human. An oral dosage form of 16.7 mL/day/kg body weight which is mouse dose equivalent to recommended human dose was set as middle dose, while the 8.3 mL/Day/kg body weight which is 0.5 times of the middle dose and 33.3 mL/day/kg body weight which is 2 times of the middle dose were set as low dose and high dose respectively. The doses were administered to the mice intragastrically at 1.2 mL/day for 32 days continuously and their effects were measured.

Instrument: mice cage, timer.

Reagents: pregnant mare serum.

Detection methods: the test substances were administered to male mice once/day for continuous 32 days. The male mice were injected with 0.2 ml (10 IU) of 50 IU/ml pregnant mare serum intramuscularly 48 hours before the test for synchronizing their sexual cycle. The male mice were housed with female mice in same cages 30 min after the last administration of test substance, and their behaviors were observed in dark. The mating latency which was calculated as time-period from housing to the first mating and mating frequency within 20 min were recorded.

### Results

### 1. Mating Latency in Mice

The effects of the compositions on the mating latency in mice were shown in Table 8.

TABLE 8
Effects of the fermented Compositions A, B and
C and the corresponding compositions without fermentation
on the mating latency in mice (X ± SD)

| | | Number | |
|---|---|---|---|
| | | of | Mating |
| Groups | | Animals | latency (s) |
| | Control | 10 | 688.42 ± 60.39 |
| Fermented | A₁ Unfermented | 10 | 615.00 ± 42.32* |
| and | High-Dose Group | | |
| unfermented | A₂ Unfermented | 10 | 627.11 ± 26.05* |
| Composition | Middle-Dose Group | | |
| A | A₃ Unfermented | 10 | 608.50 ± 19.13* |
| | Low-Dose Group | | |
| | A₄ Fermented | 10 | 570.30 ± 40.96*^{▴} |
| | High-Dose Group | | |
| | A₅ Fermented | 10 | 598.60 ± 33.88* |
| | Middle-Dose Group | | |
| | A₆ Fermented | 10 | 601.10 ± 41.48* |
| | Low-Dose Group | | |
| Fermented | B₁ Unfermented | 10 | 592.15 ± 36.54* |
| and | High-Dose Group | | |
| unfermented | B₂ Unfermented | 10 | 604.50 ± 28.15* |
| Composition | Middle-Dose Group | | |
| B | B₃ Unfermented | 10 | 618.36 ± 42.34* |
| | Low-Dose Group | | |
| | B₄ Fermented | 10 | 516.60 ± 41.26*^{▴} |
| | High-Dose Group | | |
| | B₅ Fermented | 10 | 531.53 ± 25.36*^{▴} |
| | Middle-Dose Group | | |
| | B₆ Fermented | 10 | 593.62 ± 28.14* |
| | Low-Dose Group | | |
| Fermented | C₁ Unfermented | 10 | 564.30 ± 35.18* |
| and | High-Dose Group | | |
| unfermented | C₂ Unfermented | 10 | 582.15 ± 42.85* |
| Composition | Middle-Dose Group | | |
| C | C₃ Unfermented | 10 | 605.80 ± 28.19* |
| | Low-Dose Group | | |
| | C₄ Fermented | 10 | 496.48 ± 26.72*^{▴} |
| | High-Dose Group | | |
| | C₅ Fermented | 10 | 510.20 ± 36.86*^{▴} |
| | Middle-Dose Group | | |
| | C₆ Fermented | 10 | 538.60 ± 42.18*^{▴} |
| | Low-Dose Group | | |

*Significant difference compared with the control group (P < 0.05).
^{▴}Significant difference between the fermented and unfermented compositions at the same concentrations (P < 0.05).

The results (Table 8) show that the mating latency was shortened by all of the fermented Compositions A, B and C and the unfermented Compositions A, B and C at all doses, with the fermented Composition A at high dose, the fermented Composition B at high dose, and the fermented Composition C at high, middle and low doses acting superior to the corresponding compositions at the corresponding doses without fermentation.

### 2. Mating Frequency in Mice

TABLE 9
Effects of the fermented Compositions A, B and
C and the corresponding compositions without fermentation
on mating frequency in mice (X ± SD)

| | | |
|---|---|---|
| | Number | |
| | of | Mating |
| Groups | Animals | frequency |

| | | | |
|---|---|---|---|
| | Control | 10 | 2.92 ± 1.17 |
| Fermented | A₁ Unfermented | 10 | 4.18 ± 0.98* |
| and | High-Dose Group | | |
| unfermented | A₂ Unfermented | 10 | 3.91 ± 1.38 |
| Composition | Middle-Dose Group | | |
| A | A₃ Unfermented | 10 | 3.42 ± 1.08 |
| | Low-Dose Group | | |
| | A₄ Fermented | 10 | 4.36 ± 1.21* |
| | High-Dose Group | | |
| | A₅ Fermented | 10 | 4.27 ± 1.56* |
| | Middle-Dose Group | | |
| | A₆ Fermented | 10 | 3.91 ± 1.14 |
| | Low-Dose Group | | |
| Fermented | B₁ Unfermented | 10 | 4.52 ± 1.06* |
| and | High-Dose Group | | |
| unfermented | B₂ Unfermented | 10 | 4.21 ± 1.21* |
| Composition | Middle-Dose Group | | |
| B | B₃ Unfermented | 10 | 4.08 ± 1.35* |
| | Low-Dose Group | | |
| | B₄ Fermented | 10 | 4.78 ± 1.42* |
| | High-Dose Group | | |
| | B₅ Fermented | 10 | 4.56 ± 1.05* |
| | Middle-Dose Group | | |
| | B₆ Fermented | 10 | 4.32 ± 1.25* |
| | Low-Dose Group | | |
| Fermented | C₁ Unfermented | 10 | 4.64 ± 0.96* |
| and | High-Dose Group | | |
| unfermented | C₂ Unfermented | 10 | 4.46 ± 1.34* |
| Composition | Middle-Dose Group | | |
| C | C₃ Unfermented | 10 | 4.25 ± 1.52* |
| | Low-Dose Group | | |
| | C₄ Fermented | 10 | 4.96 ± 1.02* |
| | High-Dose Group | | |
| | C₅ Fermented | 10 | 4.87 ± 1.41* |
| | Middle-Dose Group | | |
| | C₆ Fermented | 10 | 4.65 ± 1.35* |
| | Low-Dose Group | | |

*Significant difference compared with the control group (P < 0.05).
▴ Significant difference between the fermented and unfermented compositions at the same concentrations (P < 0.05).

The results (Table 9) shows that the mating frequency was increased by all of the fermented Composition A at high and middle doses, the fermented Compositions B and C at all doses, the unfermented Composition A at high dose, the unfermented Compositions B and C at all doses.

### CONCLUSION

The compositions made of maca, fructus lycii and fructus rubi fermented by probiotics were able to improve sexual function, and the fermentation with probiotics was able to enhance the sexual function-improving activities of the compositions made of maca, fructus lycii and fructus rubi.

1. A probiotic-fermented maca composition, wherein the composition is obtained by
extracting the maca, fructus lycii and fructus rubi with water,
hydrolyzing the extracts enzymatically, and
subjecting the hydrolysate to a probiotic-fermentation;
wherein said composition comprises:
1.0-10% (g/ml) of maca,
2.0-8.0% (g/ml) of fructus lycii, and
2.0-8.0% (g/ml) of fructus rubi,
wherein the enzyme used in the enzymatic hydrolysis is a high temperature amylase; and
wherein the probiotics are Bifidobacterium and Lactobacillus.

2. The composition according to claim 1, wherein said composition comprises:
3.0% (g/ml) of maca,
3.0% (g/ml) of fructus lycii, and
3.0% (g/ml) of fructus rubi.

3. The composition according to claim 1, wherein
said Bifidobacterium is selected from Bifidobacterium bifidum, Bifidobacterium breve, Bifidobacterium adolescentis or Bifidobacterium infantis; and
said Lactobacillus is selected from Lactobacillus acidophilus, Lactobacillus delbrueckii, Lactobacillus casei, Lactobacillus rhamnosus or Lactobacillus plantarum.

4. The composition according to claim 1,
wherein the composition further comprises sweeteners which are
sucrose which is present in an amount of 3.0% (g/ml), and
sucralose which is present in an amount of 0.008% (g/ml).

5. A method for preparing the composition according to claim 1, wherein the method comprises the steps of:
(1) obtaining a mixed powder of maca, fructus lycii and fructus rubi by
grinding the maca to a powder with a fineness of 100 mesh or more, grinding the mixture of the fructus lycii and fructus rubi to a powder with a fineness of 10 mesh or more, and
mixing the powders of the maca, fructus lycii and fructus rubi together;
(2) obtaining a complex extract of the maca, fructus lycii and fructus rubi by
immersing the mixed powder obtained in the above step (1) in water weighed 10-16 times greater than the mixed powder for 30 min to obtain a mixture,
extracting the mixture at around 115° C. under high-pressure for 40 minutes to obtain an extract,
hydrolyzing the extract with a high-temperature amylase after cooling to 85-90° C. and adjusting the pH to around 5.5,
filtering and concentrating the hydrolysate under reduced pressure after sterilization to obtain a filtrate from which insoluble residues have been removed, and
adding into the filtrate an amount of glucose and inorganic salts which is followed by adjustment of pH to 6.5-7.0 and sterilization; and
(3) obtaining a probiotic-fermented maca composition by a probiotic-fermentation which comprises:
seeding 0.5% (v/v) of one or two or more pre-cultured Bifidobacterium into the complex extract obtained in the above step (2) when it is cooled to about 39° C. and culturing for about 3 to 8 hours,
seeding thereto further 0.5% (v/v) of one or two or more pre-cultured Lactobacillus and culturing for about 20 to 22 hours,
decreasing the culture temperature to 28° C. when the pH is dropped to 4.2 or less, and
continuing the fermentation until the pH is dropped to 4.0 and stabilizing the resultant ferment.

6. The method according to claim 5, wherein said composition comprises:
1.0-10% (g/ml) of maca,
2.0-8.0% (g/ml) of fructus lycii, and
2.0-8.0% (g/ml) of fructus rubi.

7. The method according to claim 5, wherein
the ratio between the high-temperature amylase and the mixed powder in the step (2) is 30-40 U/g;
the glucose is added into the filtrate in an amount of 0.5% (g/ml); and
the inorganic salts are
5.0 mg/100 ml of FeSO₄.7H₂O,
4.4 mg/100 ml of ZnSO₄.7H₂O, and
50 mg/100 ml of MgSO₄.7H₂O.

8. The method according to claim 5, wherein the method further comprises, after the enzymatic hydrolysis and filtration in the step (2), adding sweeteners which are
3.0 g/100 ml of sucrose, and
0.008 g/100 ml of sucralose.

9. The method according to claim 5, wherein in the step (3),
said Bifidobacterium is selected from Bifidobacterium bifidum, Bifidobacterium breve, Bifidobacterium adolescentis or Bifidobacterium infantis; and
said Lactobacillus is selected from Lactobacillus acidophilus, Lactobacillus delbrueckii, Lactobacillus casei, Lactobacillus rhamnosus or Lactobacillus plantarum.

10. Use of the composition according to claim 1, in the manufacture of a health product, food or beverage for improving fatigue tolerance and sexual function in a subject.

11. The composition according to claim 2, wherein the composition further comprises sweeteners which are
a) sucrose which is present in an amount of 3.0% (g/ml), and
b) sucralose which is present in an amount of 0.008% (g/ml).

12. The composition according to claim 3, wherein the composition further comprises sweeteners which are
a) sucrose which is present in an amount of 3.0% (g/ml), and
b) sucralose which is present in an amount of 0.008% (g/ml).

13. The method according to claim 6, wherein in the step (3),
a) said Bifidobacterium is selected from Bifidobacterium bifidum, Bifidobacterium breve, Bifidobacterium adolescentis or Bifidobacterium infantis; and
b) said Lactobacillus is selected from Lactobacillus acidophilus, Lactobacillus delbrueckii, Lactobacillus casei, Lactobacillus rhamnosus or Lactobacillus plantarum.

14. The method according to claim 7, wherein in the step (3),
a) said Bifidobacterium is selected from Bifidobacterium bifidum, Bifidobacterium breve, Bifidobacterium adolescentis or Bifidobacterium infantis; and
b) said Lactobacillus is selected from Lactobacillus acidophilus, Lactobacillus delbrueckii, Lactobacillus casei, Lactobacillus rhamnosus or Lactobacillus plantarum.

15. The method according to claim 8, wherein in the step (3),
a) said Bifidobacterium is selected from Bifidobacterium bifidum, Bifidobacterium breve, Bifidobacterium adolescentis or Bifidobacterium infantis; and
b) said Lactobacillus is selected from Lactobacillus acidophilus, Lactobacillus delbrueckii, Lactobacillus casei, Lactobacillus rhamnosus or Lactobacillus plantarum.

16. Use of the composition according to claim 2, in the manufacture of a health product, food or beverage for improving fatigue tolerance and sexual function in a subject.

17. Use of the composition according to claim 3, in the manufacture of a health product, food or beverage for improving fatigue tolerance and sexual function in a subject.

18. Use of the composition according to claim 4, in the manufacture of a health product, food or beverage for improving fatigue tolerance and sexual function in a subject.

19. The composition according to claim 1, wherein the composition is obtained by
a) extracting the maca, fructus lycii and fructus rubi with water,
b) hydrolyzing the extracts enzymatically, and
c) subjecting the hydrolysate to a probiotic-fermentation;
wherein said composition of maca, fructus lycii and fructus rubi is comprised of:
i) 3.0% (g/ml) of maca,
ii) 3.0% (g/ml) of fructus lycii, and
iii) 3.0% (g/ml) of fructus rubi,
wherein the enzyme used in the enzymatic hydrolysis is a high temperature amylase; and the probiotics are Bifidobacterium and Lactobacillus and wherein:
a) said Bifidobacterium is selected from Bifidobacterium bifidum, Bifidobacterium breve, Bifidobacterium adolescentis or Bifidobacterium infantis; and
b) said Lactobacillus is selected from Lactobacillus acidophilus, Lactobacillus delbrueckii, Lactobacillus casei, Lactobacillus rhamnosus or Lactobacillus plantarum;
wherein the composition further comprises sweeteners which are
a) sucrose which is present in an amount of 3.0% (g/ml), and
b) sucralose which is present in an amount of 0.008% (g/ml).

20. A method according to claim 5 for preparing the composition of claim 1, wherein the method comprises the steps of:
(1) obtaining a mixed powder of maca, fructus lycii and fructus rubi by
a) grinding the maca to a powder with a fineness of 100 mesh or more, grinding the mixture of the fructus lycii and fructus rubi to a powder with a fineness of 10 mesh or more, and
b) mixing the powders of the maca, fructus lycii and fructus rubi together;
wherein said powder are in the following percents:
i) 1.0-10% (g/ml) of maca,
ii) 2.0-8.0% (g/ml) of fructus lycii, and
iii) 2.0-8.0% (g/ml) of fructus rubi
(2) obtaining a complex extract of the maca, fructus lycii and fructus rubi by
a) immersing the mixed powder obtained in the above step (1) in water weighed 10-16 times greater than the mixed powder for 30 min to obtain a mixture,
b) extracting the mixture at around 115° C. under high-pressure for 40 minutes to obtain an extract,
c) hydrolyzing the extract with a high-temperature amylase after cooling to 85-90° C. and adjusting the pH to around 5.5,
d) filtering and concentrating the hydrolysate under reduced pressure after sterilization to obtain a filtrate from which insoluble residues have been removed, and
e) adding into the filtrate an amount of glucose and inorganic salts which is followed by adjustment of pH to 6.5-7.0 and sterilization; and
wherein;
i) the ratio between the high-temperature amylase and the mixed powder in the step (2) is 30-40 U/g;
ii) the glucose is added into the filtrate in an amount of 0.5% (g/ml); and
iii) the inorganic salts are
1) 5.0 mg/100 ml of FeSO₄.7H₂O,
2) 4.4 mg/100 ml of ZnSO₄.7H₂O, and
3) 50 mg/100 ml of MgSO₄.7H₂O
wherein after the enzymatic hydrolysis and filtration in the step (2), adding sweeteners which are:
a) 3.0 g/100 ml of sucrose, and
b) 0.008 g/100 ml of sucralose,
followed by;
(3) obtaining a probiotic-fermented maca composition by a probiotic-fermentation which comprises:
a) seeding 0.5% (v/v) of one or two or more pre-cultured Bifidobacterium into the complex extract obtained in the above step (2) when it is cooled to about 39° C. and culturing for about 3 to 8 hours,
b) seeding thereto further 0.5% (v/v) of one or two or more pre-cultured Lactobacillus and culturing for about 20 to 22 hours,
c) decreasing the culture temperature to 28° C. when the pH is dropped to 4.2 or less, and
d) continuing the fermentation until the pH is dropped to 4.0 and stabilizing the resultant ferment,
wherein;
i) said Bifidobacterium is selected from Bifidobacterium bifidum, Bifidobacterium breve, Bifidobacterium adolescentis or Bifidobacterium infantis; and
ii) said Lactobacillus is selected from Lactobacillus acidophilus, Lactobacillus delbrueckii, Lactobacillus casei, Lactobacillus rhamnosus or Lactobacillus plantarum.
